(19) United States
(12) Patent Application Publication
(10) Pub. No.: US 2018/0326186 A1
(43) Pub. Date: Nov. 15, 2018

(54) EXPANDABLE SHEATH

(71) Applicant: Boston Scientific Scimed, Inc., Maple Grove, MN (US)

(72) Inventors: Pu Zhou, Maple Grove, MN (US); Huisun Wang, Maple Grove, MN (US)

(73) Assignee: BOSTON SCIENTIFIC SCIMED, INC., Maple Grove, MN (US)

(21) Appl. No.: 16/027,866

(22) Filed: Jul. 5, 2018

Related U.S. Application Data
(63) Continuation of application No. 14/174,326, filed on Feb. 6, 2014.
(60) Provisional application No. 61/762,870, filed on Feb. 9, 2013.

(51) Int. Cl. A61M 25/09; A61M 25/00; A61M 25/06; A61M 39/06

(52) U.S. Cl. CPC A61M 25/09; A61M 25/0074; A61M 25/0045; A61M 25/0071; A61M 2025/0024; A61M 25/0043; A61M 25/005; A61M 39/06; A61M 25/0662

(57) ABSTRACT

A medical device assembly may include an elongated tubular membrane having a wall defining a lumen extending through the membrane from a proximal end to a distal end, the lumen having a first inner diameter, and a percutaneous medical device having a maximum outer diameter greater than the first inner diameter, wherein the membrane is configured to permit the medical device to pass through the lumen, and wherein the membrane includes a plurality of longitudinally-oriented channels recessed along an inner surface of the wall. A medical device delivery sheath may include a tubular first layer of polymeric material formed into a wavy cross-section having a plurality of lobes and a plurality of valleys, wherein the first layer of material is resiliently expandable in a radial direction from a relaxed configuration to an expanded configuration, and wherein the first layer of material is substantially non-expandable in an axial direction.

### CROSS-REFERENCE TO RELATED APPLICATIONS

This application is a continuation of U.S. patent application Ser. No. 14/174,326, filed Feb. 6, 2014 which claims priority to U.S. Provisional Application No. 61/762,870 filed Feb. 9, 2013.

### TECHNICAL FIELD

The invention relates generally to medical devices and more particularly to medical devices that are adapted for use in percutaneous medical procedures.

### BACKGROUND

Some percutaneous procedures can involve relatively large, bulky medical devices that must be advanced through relatively narrow and tortuous vasculature. Such advancement may result in peripheral damage to the wall of the vessel, particularly when the medical device must traverse a sharp bend in the vessel, due to the shear force exerted by the medical device against the vessel wall. A continuing need exists to reduce or eliminate the chances of injuring the vessel during percutaneous medical procedures.

### SUMMARY

A medical device assembly may include an elongated guidewire, an elongated tubular membrane disposed about the guidewire, the membrane having a wall defining a lumen extending through the membrane from a proximal end to a distal end, the lumen having a first inner diameter, and a percutaneous medical device having a maximum outer diameter greater than the first inner diameter, wherein the membrane is configured to permit the percutaneous medical device to pass through the lumen, and wherein the membrane includes a plurality of longitudinally-oriented channels recessed along an inner surface of the wall.

A medical device delivery sheath may include a tubular first layer of polymeric material formed into a wavy cross-section having a plurality of lobes and a plurality of valleys between adjacent lobes, wherein the tubular first layer of polymeric material is resiliently expandable in a radial direction from a relaxed configuration to an expanded configuration, wherein the tubular first layer of polymeric material has a first outer radial extent as measured from a central longitudinal axis in the relaxed configuration, and a second outer radial extent as measured from the central longitudinal axis in the expanded configuration, wherein the second outer radial extent is greater than the first outer radial extent, and wherein the tubular first layer of polymeric material is substantially non-expandable in an axial direction.

Although discussed with specific reference to use within the vasculature of a patient, medical devices and methods of use in accordance with the disclosure can be adapted and configured for use in other parts of the anatomy, such as the digestive system, the respiratory system, or other parts of the anatomy of a patient.

### BRIEF DESCRIPTION OF THE FIGURES

FIG. 1 is a partial schematic view of an example membrane disposed within a vessel;

FIG. 1A is a cross-sectional view of FIG. 1 taken along the line 1A-1A;

FIG. 2 is a partial schematic view of the example membrane of FIG. 1 including a medical device disposed therein;

FIG. 2A is a cross-sectional view of FIG. 2 taken along the line 2A-2A;

FIG. 2B is a cross-sectional view of FIG. 2 taken along the line 2B-2B;

FIG. 2C is a cross-sectional view of FIG. 2 taken along the line 2C-2C;

FIG. 3A is a cross-sectional view of a portion of an example membrane in a relaxed configuration;

FIG. 3B is a cross-sectional view of a portion of the example membrane of FIG. 3A is an expanded configuration;

FIG. 4A is a cross-sectional view of a portion of an example membrane in a relaxed configuration;

FIG. 4B is a cross-sectional view of a portion of the example membrane of FIG. 4A is an expanded configuration;

FIG. 5A is a cross-sectional view of a portion of an example membrane in a relaxed configuration;

FIG. 5B is a cross-sectional view of a portion of the example membrane of FIG. 5A is an expanded configuration;

FIG. 6 is a perspective view of a portion of an example membrane;

FIG. 7 is a perspective view of a portion of an example membrane;

FIG. 8A is a side view of an example membrane;

FIG. 8B is a side view of the example membrane of FIG. 8A including a medical device partially advanced therethrough;

FIG. 8C is a side view of the example membrane of FIG. 8A including a medical device partially advanced therethrough;

FIG. 9A is a partial cross-sectional view of an example membrane including a medical device being withdrawn therethrough; and

FIG. 9B is a partial cross-sectional view of an example membrane including a medical device being withdrawn therethrough.

While the invention is amenable to various modifications and alternative forms, specifics thereof have been shown by way of example in the drawings and will be described in greater detail below. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range, including the endpoints (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangable with each other to form other additional embodiments or to complement and/or enrich the described embodiment(s), as would be understood by one of ordinary skill in the art.

Some percutaneous medical procedures may require relatively large and/or bulky medical devices (18+ French in size) to be inserted through a patient's vasculature. In some cases, those medical devices may pass through a tortuous and/or peripheral vessel. As the medical device is navigated through the vasculature, the vessel wall may be subjected to a shear force applied by the medical device as the medical device moves through the vessel lumen and makes contact with the vessel wall. The shear force may cause injury to the vessel in tortuous portions of the vasculature as the medical device is forced to make turns, or when the medical device travels through calcified or diseased vessels.

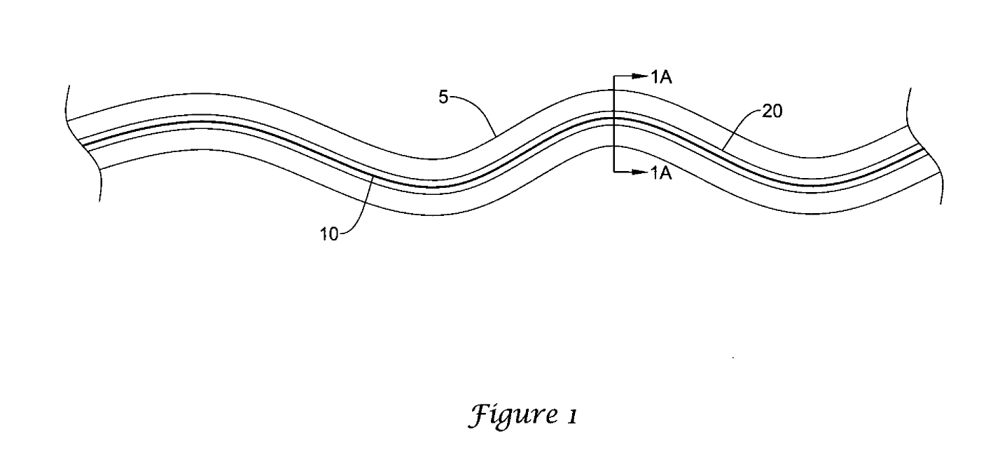

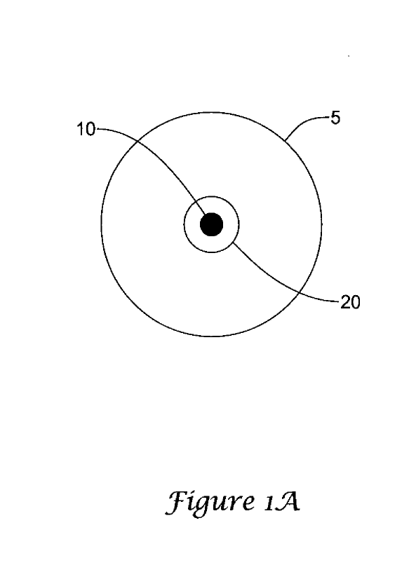

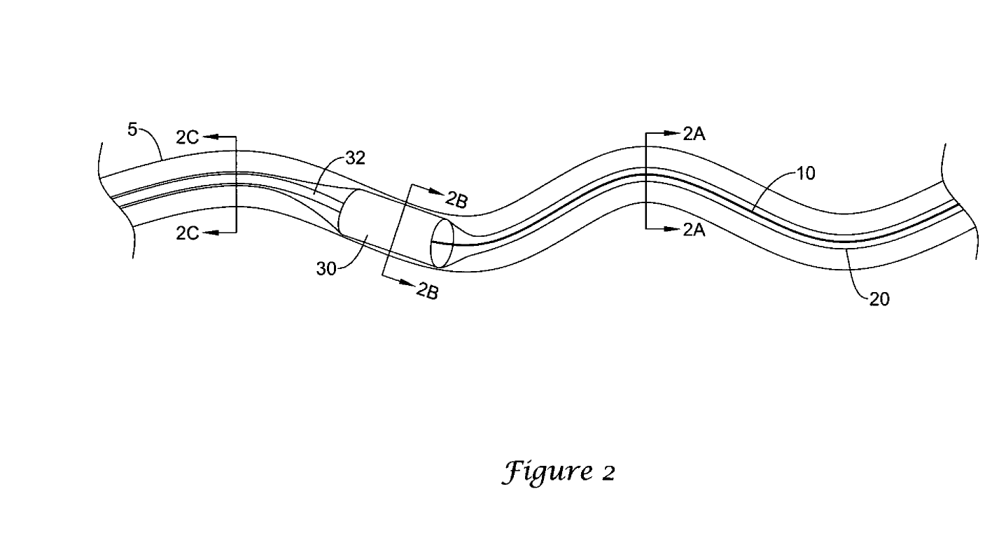

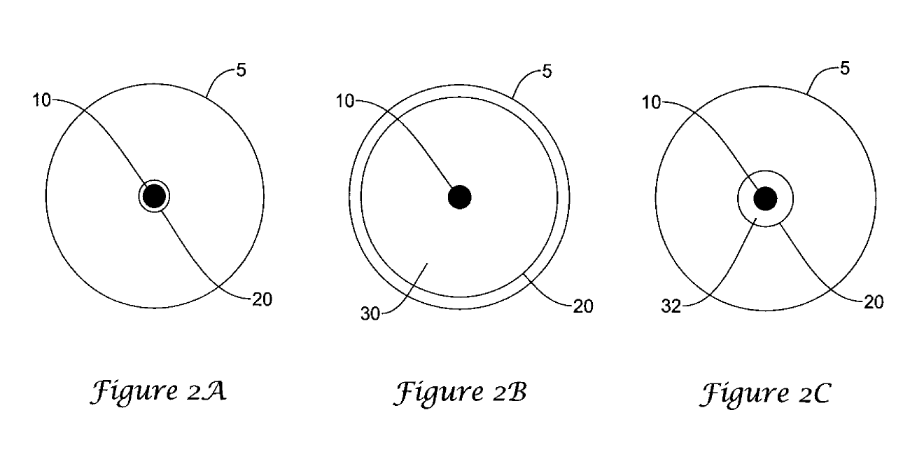

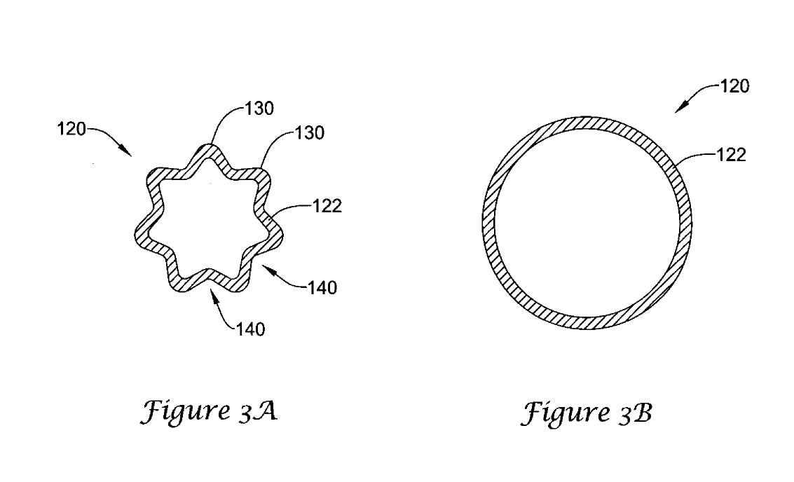

The risk of injury from the shear force applied against the vessel wall by a traversing medical device may be reduced by protecting the vessel wall from the shear force. It may also be desirable for a delivery sheath to maintain a smaller profile while permitting expansion to accommodate the passage of a medical device therethrough. FIG. 1 illustrates a schematic view of a vessel 5 having a guidewire 10 disposed therein. An example membrane or medical device delivery sheath 20, which may be an elongated tubular membrane made from a highly flexible elastic material, or having a highly flexible elastic structure, is shown disposed about the guidewire 10. The membrane 20 is schematically illustrated in FIGS. 1-2C as having a substantially annular shape. However, the skilled artisan will recognize that other shapes and/or configurations are possible within the scope of the present disclosure, as will be apparent from the discussion below, and other shapes or configurations discussed herein may be used in the configuration(s) schematically shown in FIGS. 1-2C. An example membrane 20 in accordance with the present disclosure may include none, one, some, or all of the features shown in FIGS. 3A-9B.

In general, the membrane 20 may be described as having an elongated tubular structure having a lumen extending therethrough from a proximal end to a distal end. The membrane 20 may include a wall having an inner surface and an outer surface. In some embodiments, a thickness of the wall may be defined by the inner surface and the outer surface.

The membrane 20 and/or the lumen may be configured to radially expand and/or contract between a relaxed condition and an expanded condition. In the relaxed condition, the lumen may have a first inner diameter defined by the inner surface of the wall. In some embodiments, as will be apparent herein, the first inner diameter may instead be defined as a first inner radial extent or distance from a central longitudinal axis of the membrane 20. In the expanded condition, the lumen may have a second inner diameter defined by the inner surface of the wall. In some embodiments, as will be apparent herein, the second inner diameter may instead be defined as a second inner radial extent or distance from a central longitudinal axis of the membrane 20. In some embodiments, the second inner diameter may be greater than the first inner diameter. Similarly, the second inner radial extent may be greater than the first inner radial extent.

Similarly, the membrane 20 may have an outer diameter or outer radial extent defined by the outer surface of the wall. In the relaxed condition, the membrane 20 may have a first outer diameter or first outer radial extent defined by the outer surface of the wall. In the expanded condition, the membrane 20 may have a second outer diameter or a second outer radial extent defined by the outer surface of the wall. In some embodiments, the second outer diameter may be greater than the first outer diameter. Similarly, the second outer radial extent may be greater than the first outer radial extent.

In some embodiments, the membrane 20 may be configured to permit the lumen to radially expand to the second inner diameter or the second inner radial extent. In some embodiments, the membrane 20 is configured to substantially prevent axial stretching along the lumen. In other words, the membrane 20 may permit the lumen to expand radially outward without stretching or expanding in an axial direction. In some embodiments, the second inner diameter or the second inner radial extent may be greater than the first outer diameter or the first outer radial extent.

In some embodiments, this behavior or characteristic may be facilitated by the shape of the membrane in the relaxed condition and/or the yield strain of the material forming the membrane. Yield strain may be described as a maximum amount of strain a material may be subjected to with no permanent deformation. In other words, as long as the material under strain has not reached its yield strain, it will elastically recover to its original shape when the strain or force is removed.

For example, the membrane 120 illustrated in cross-section in FIGS. 3A-3B may be formed into a first layer of material 122 including a profile having a wavy pattern or cross-sectional shape including a plurality of lobes 130. When the membrane 120 is formed into a profile having a wavy pattern or cross-sectional shape, the membrane 120 may be expanded to a radial extent greater than its yield strain would allow if the membrane 120 were formed into a round cross-sectional shape having a relaxed radial extent similar to that of the wavy cross-sectional shape. The profile may include a first inner radial extent corresponding to an innermost extent of the plurality of lobes 130 and a first outer radial extent corresponding to the outermost extent or tips of the plurality of lobes 130. A plurality of valleys 140 may be formed between adjacent lobes 130 in the relaxed condition, wherein one valley 140 is disposed between two adjacent lobes 130, as seen in FIG. 3A. In the expanded condition, the plurality of valleys 140 may be translated radially outward relative to the relaxed condition. Once the plurality of valleys 140 reach a common radial extent with the tips of the plurality of lobes 130, the plurality of valleys 140 and the plurality of lobes 130 may translate radially outward together in unison to define the second inner radial extent and/or the second outer radial extent, as seen in FIG. 3B. In some embodiments, the second inner radial extent may be greater than the first outer radial extent. In some embodiments, for example, the first outer radial extent may be about 14 F and the second outer radial extent may be about 22 F. In some embodiments, after removing the strain, the membrane 120 may recover to the first outer radial extent. In some embodiments, after removing the strain, the membrane 120 may recover to a third outer radial extent greater than the first outer radial extent and less than the second outer radial extent. For example, the membrane 120 may recover to a third outer radial extent of about 16 F. The preceding expansion and recovery behavior discussed herein (i.e., exhibited by the membrane 120) may occur in any embodiment of a membrane embraced by the present disclosure. In general, a first layer having a higher quantity of valleys and lobes present in the relaxed condition may permit a greater amount of expansion before encountering permanent deformation.

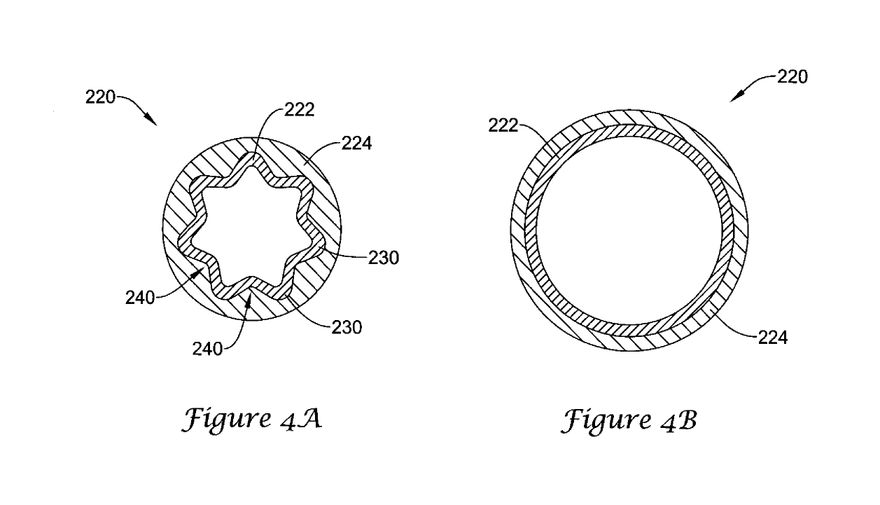

In some embodiments, a membrane may be configured as shown in FIGS. 4A-4B. As illustrated in FIGS. 4A-4B, a membrane 220 may include a first layer 222 and a second layer 224 disposed about the first layer 222. In some embodiments, the first layer 222 and the second layer 224 may be mechanically or adhesively attached or joined to each other. In some embodiments, the first layer 222 and the second layer 224 may be co-extruded to form a unitary structure wherein the first layer 222 and the second layer 224 are fused and/or comingled at a molecular level.

In some embodiments, the first layer 222 may include a profile having a wavy pattern or cross-sectional shape including a plurality of lobes 230. A plurality of valleys 240 may be formed between adjacent lobes 230 in the relaxed condition, wherein one valley 240 is disposed between two adjacent lobes 230, as seen in FIG. 4A. In the expanded condition, the plurality of valleys 240 may be translated radially outward relative to the relaxed condition. Once the plurality of valleys 240 reach a common radial extent with the tips of the plurality of lobes 230, the plurality of valleys 240 and the plurality of lobes 230 may translate radially outward together in unison to define the second inner radial extent, as seen in FIG. 4B. The second layer 224 may be disposed about the first layer 222. The second layer 224 may form a generally smooth, generally uniform outer extent. The second layer 224 may expand along with the first layer 222, to a smaller degree while the plurality of valleys 240 is translated outward to the common radial extent and then to a larger degree concurrently with the first layer 222, when the first layer 222 and the second layer 224 are substantially coaxial. In some embodiments, the membrane 220 may expand radially outward only until the plurality of valleys 240 reaches the common radial extent with the tips of the plurality of lobes 230. In general, the second layer 224 may be a different material than the first layer 222, and in some embodiments, the second layer 224 may have a higher yield strain than the first layer 222.

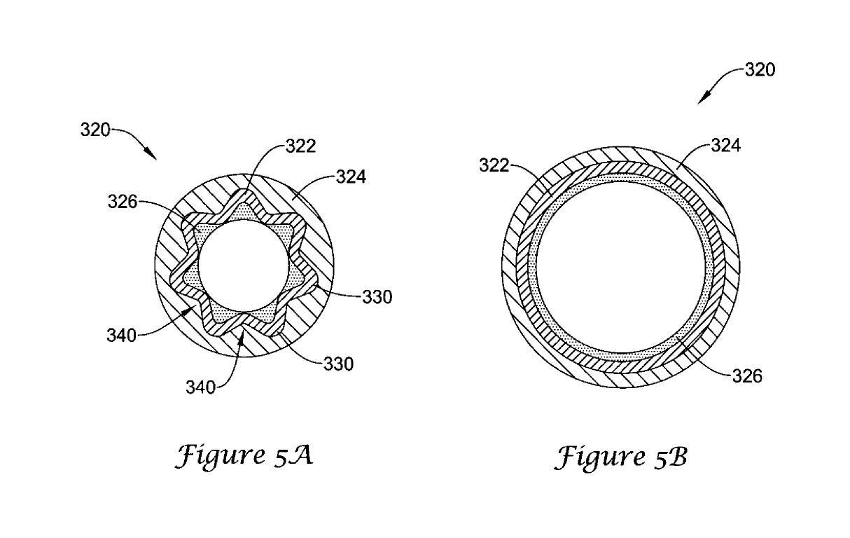

In some embodiments, a membrane may be configured as shown in FIGS. 5A-5B. As illustrated in FIGS. 5A-5B, a membrane 320 may include a first layer 322, a second layer 324 disposed about the first layer 322, and a third layer 326, wherein the first layer 322 is disposed about the third layer 326 (i.e. the third layer 326 is an innermost layer). In some embodiments, the first layer 322, the second layer 324, and/or the third layer 326 may be mechanically or adhesively attached or joined to each other. In some embodiments, the first layer 322, the second layer 324, and/or the third layer 326 may be co-extruded to form a unitary structure wherein the first layer 322, the second layer 324, and/or the third layer 326 are comingled and/or fused at a molecular level. In some embodiments, two of the three layers may be fused or comingled, and the other layer may be adhesively or mechanically joined to the two fused layers.

In some embodiments, the first layer 322 may include a profile having a wavy pattern or cross-sectional shape including a plurality of lobes 330. A plurality of valleys 340 may be formed between adjacent lobes 330 in the relaxed condition, wherein one valley 340 is disposed between two adjacent lobes 330, as seen in FIG. 5A. In the expanded condition, the plurality of valleys 340 may be translated radially outward relative to the relaxed condition. Once the plurality of valleys 340 reach a common radial extent with the tips of the plurality of lobes 330, the plurality of valleys 340 and the plurality of lobes 330 may translate radially outward together in unison. The second layer 324 may be disposed about the first layer 322. The second layer 324 may form a generally smooth, generally uniform outer extent. The second layer 324 may expand along with the first layer 322, to a smaller degree while the plurality of valleys 340 is translated outward to the common radial extent and then to a larger degree concurrently with the first layer 322, when the first layer 322 and the second layer 324 are substantially coaxial. The third layer 326 may define the inner radial extent, as seen in FIGS. 5A and 5B. The third layer 326 may form a generally smooth, generally uniform inner radial extent. The third layer 326 may expand along with the first layer 322 while the plurality of valleys 340 is translated outward to the common radial extent and then concurrently with the first layer 322 and the second layer 324, when the first layer 322, the second layer 324, and the third layer 326 are substantially coaxial. In some embodiments, the membrane 320 may expand radially outward only until the plurality of valleys 340 reaches the common radial extent with the tips of the plurality of lobes 330. In general, the second layer 324 and/or the third layer 326 may be formed of a different material than the first layer 322. In some embodiments, the second layer 324 and the third layer 326 may be formed of a different material, or the second layer 324 and the third layer 326 may be formed of the same material. In some embodiments, the second layer 324 and the third layer 326 may have a higher yield strain than the first layer 322.

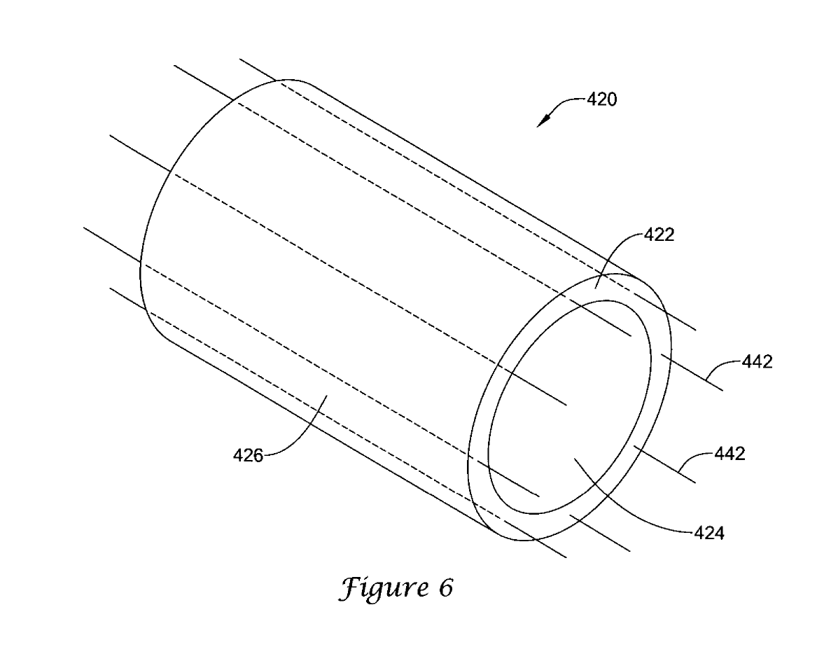

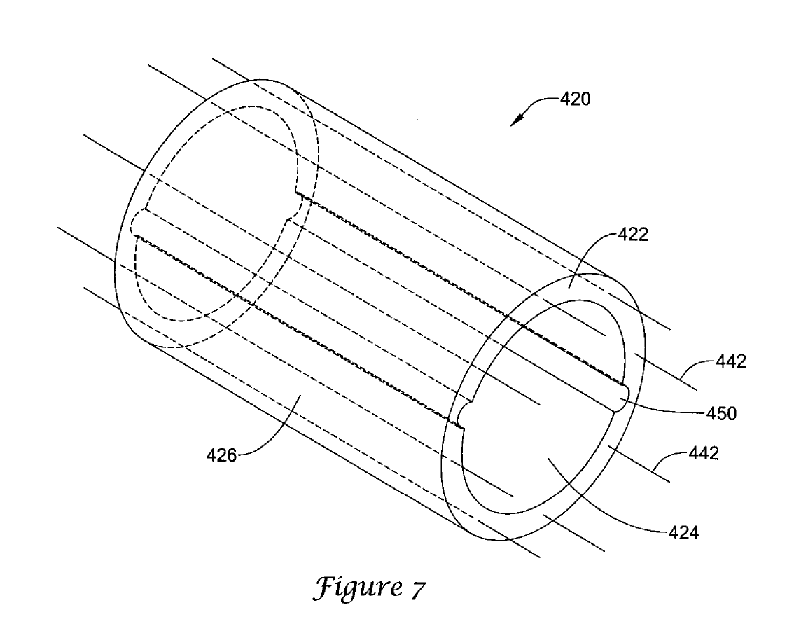

In some embodiments, for example, as illustrated in FIGS. 6-7, to facilitate the radially expanding behavior or characteristic, a membrane 420 may be formed to include a plurality of filaments or fibers 442 oriented axially, or generally parallel to the lumen or a central axis of the lumen and/or membrane 420. In some embodiments, the plurality of filaments or fibers 442 may each be longitudinally aligned with the lumen. In some embodiments, the plurality of filaments or fibers 442 may be embedded within the wall 422 of the membrane 420. Although not expressly illustrated, in some embodiments, the plurality of filaments or fibers 442 may be disposed on the inner surface 424 or on the outer surface 426 of the wall 422. The plurality of filaments or fibers 442 may exhibit high non-compliance in an axial direction, and may be highly compliant or flexible in a tangential (i.e., transverse or radial) direction relative to the central longitudinal axis of the lumen.

In some embodiments, the plurality of filaments or fibers 442 may include two, three, four, five, six, seven, eight, ten, twelve, fifteen, or more individual fibers. In some embodiments, the plurality of filaments or fibers 442 may be spaced or arranged equally about a circumference of the membrane 420 (i.e., angularly equidistant about a central longitudinal axis). In some embodiments, the plurality of filaments or fibers 442 may be spaced or arranged unequally about a circumference of the membrane 420 (i.e., not angularly equidistant about a central longitudinal axis). In some embodiments, the plurality of filaments or fibers 442 may be formed in a suitable shape or cross-section, including but not limited to, round, rectangular, square, triangular, tubular, ovoid, other polygonal shapes, or other suitable shapes or cross-sections. In some embodiments, the plurality of filaments or fibers 442 may be formed from a material having a high flexural modulus compared to the surrounding wall 422 of the membrane 420. For example, the plurality of filaments or fibers 442 may be formed from a material having a flexural modulus greater than 100 MPa, greater than 250 MPa, greater than 400 MPa, greater than 500 MPa, greater than 600 MPa, or more. Additionally, while not expressly illustrated, the membrane(s) illustrated in FIGS. 1-2C and 6-9B may include the profile(s), or be formed using the construction(s), illustrated in FIGS. 3A-5B, in accordance with the present disclosure.

In some embodiments, the inner surface 424 of the wall 422 may include one or more layers or coatings, such as a lubricious coating, a hydrophilic coating, a hydrophobic coating, or other suitable coatings, and the like, or the membrane 420 may include a lubricant disposed within the lumen. In some embodiments, the outer surface 426 of the wall 422 may include one or more layers or coatings, such as a lubricious coating, a hydrophilic coating, a hydrophobic coating, or other suitable coating, and the like, or the membrane 420 may include a lubricant disposed upon the outer surface 426.

In some embodiments, the membrane 420 may also include a plurality of channels 450 extending longitudinally along the inner surface 424 of the wall 422, as illustrated in FIG. 7. In some embodiments, the plurality of channels 450 may be filled with a porous material permitting fluid to pass therethrough. The plurality of channels 450 may be formed in a variety of shapes, such as rounded, triangular, semicircle, U-shaped, V-shaped, etc. as desired. In some embodiments, the plurality of channels 450 may be recessed within the wall 422 of the membrane 420 from the inner surface 424. In some embodiments, the plurality of channels 450 may be formed with a depth (i.e., a distance from the inner surface 424 to a farthest radial extent of the channel from the central longitudinal axis) of about 0.0002 inches up to about 40%, about 50%, about 60%, about 75%, about 85%, or about 95% of a total thickness of the wall 422.

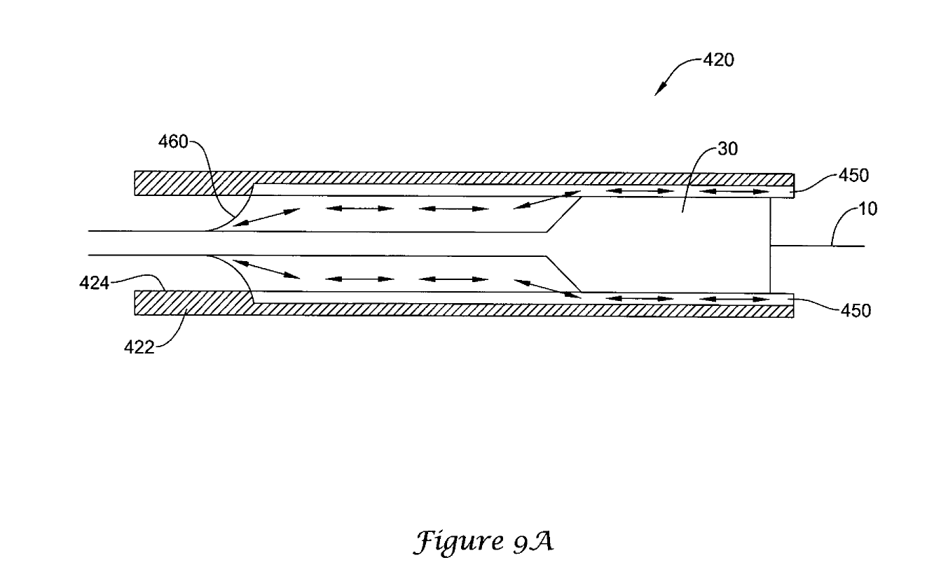

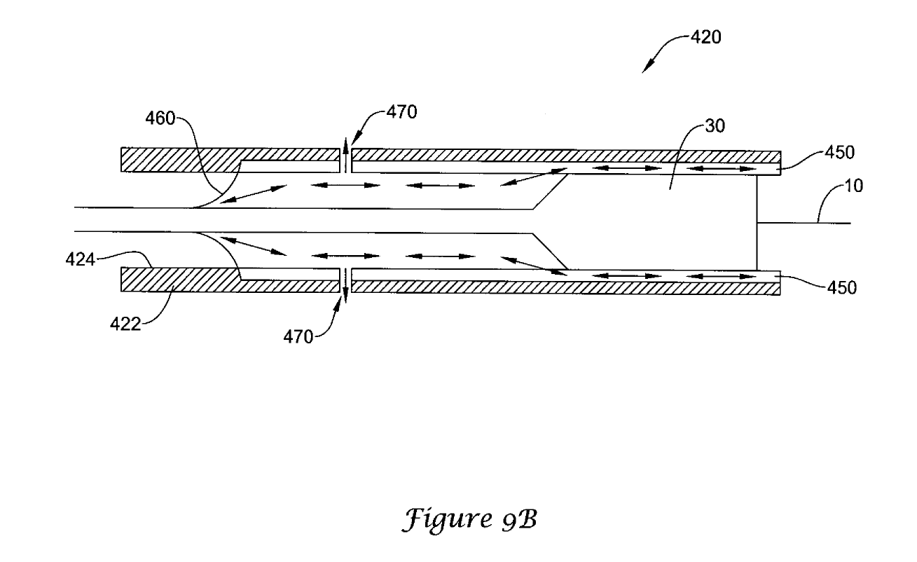

As may be seen in FIGS. 9A and 9B, the membrane 420 may also include a hemostatic valve 460 disposed within a lumen of the membrane 420 proximal of a distal end of the membrane 420. The hemostatic valve 460 may prevent blood or other bodily fluid(s) from flowing proximally through the lumen of the membrane 420. During withdrawal of the medical device 30, blood or other bodily fluid(s) may be present within a lumen of the membrane 420. Withdrawal of the medical device 30 may compress the blood or other bodily fluid(s) between the medical device 30 and the hemostatic valve 460. However, since most fluids are generally incompressible, withdrawal of the medical device 30 becomes increasingly more difficult as hydraulic pressure within the lumen of the membrane 420 increases. In some embodiments, a plurality of channels 450 may extend along the inner surface 424 of the wall 422 from a distal end of the membrane 420 proximally to the hemostatic valve 460 or to a position near the hemostatic valve 460. The plurality of channels 450 may provide pathways for blood or other bodily fluid(s) to flow in and/or out of the lumen of the membrane 420 around the medical device 30, thereby providing a means to reduce the hydraulic pressure within the lumen of the membrane 420 and/or maintain a consistent hydraulic pressure within the lumen of the membrane 420 as the medical device 30 is withdrawn into and/or through the lumen of the membrane 420.

In some embodiments, the membrane 420 may further or alternatively include one or more apertures 470 extending through the wall 422 of the membrane 420, as illustrated in FIG. 9B. The one or more apertures 470 may provide pathways for blood or other bodily fluid(s) to flow in and/or out of the lumen of the membrane 420, thereby providing a means to reduce the hydraulic pressure within the lumen of the membrane 420 and/or maintain a consistent hydraulic pressure within the lumen of the membrane 420 as the medical device 30 is withdrawn into and/or through the lumen of the membrane 420.

FIG. 2 illustrates a schematic view of an example membrane 20 disposed within a vessel 5 having a medical device 30 being advanced through a lumen of the membrane 20 along a guidewire 10. For reference, the view shown in FIG. 2 may generally be described as showing “proximal” toward the left side of FIG. 2 and “distal” toward the right side of FIG. 2. Accordingly, the medical device 30 is shown as being advanced from the proximal end or portion of the membrane 20 toward the distal end or portion of the membrane 20. With respect to a user of the membrane and/or medical device, the proximal end may be considered closest to the user (or external to a patient) and the distal end farthest from the user (or internal to a patient). However, the skilled artisan will appreciate that the orientations and/or directions may be reversed as necessary or appropriate.

The medical device 30 is schematically illustrated in FIG. 2 as a cylindrical element disposed at the distal end of an elongate shaft 32. One of ordinary skill in the art will recognize that the medical device 30 and the elongate shaft 32 may be a single object, element, or device, or may be a combination of elements that together make up a medical device suitable for insertion through the membrane 20. For example, the medical device 30 may be a single catheter or sheath having a uniform outer diameter or the medical device 30 may be an assembly having a stepped or variable outer diameter, or the medical device 30 may be some combination of these (i.e., a single catheter with a stepped or variable outer diameter).

In some embodiments, the medical device 30 may include an atherectomy device, an angioplasty device, a balloon dilatation catheter, a distal protection device, an embolic filtering device, a valvectomy device, a valvuloplasty device, a stent delivery device, a transaortic valve implantation device, an ablation device, an object retrieval device, a guide catheter or sheath, a diagnostic catheter, or other suitable device. For simplicity, the following discussion will generally refer to a medical device 30, which may or may not include the elongate shaft 32 shown in FIG. 2.

The medical device 30 may have a maximum outer diameter that may be defined as the farthest or largest radial extent from a central longitudinal axis of the medical device 30, or the maximum circumference or perimeter of the medical device 30. In some embodiments, the medical device 30 may have a maximum outer diameter of about 16 F (French), about 18 F, about 20 F, or more. In some embodiments, the medical device 30 may have a maximum outer diameter that is greater than the first inner diameter of the lumen of the membrane 20 (i.e., the inner diameter of the lumen of the membrane 20 in the radially relaxed condition) and/or the first outer diameter of the membrane 20 (i.e., the outer diameter of the membrane 20 in the radially relaxed condition).

During operation, the membrane 20 may be advanced through the vessel 5 to a treatment site. In some embodiments, the membrane 20 may be delivered via a guide or delivery catheter, or the membrane 20 may be fixedly or removably attached to a guidewire for navigation through the vasculature to the treatment site. For example, the membrane 20 may be attached along one side to a guidewire, or the membrane 20 may be attached at its distal end to a distal end of a guidewire. The guidewire may push or pull the membrane 20 through the vasculature to the treatment site. In some embodiments, the elastic nature of the membrane 20 may form a natural fit to the guidewire.

Following placement within the vessel 5, the membrane 20 may remain or be maintained in a substantially stationary position along the guidewire or relative to the vessel 5 as the medical device 30 is passed through the lumen of the membrane 20. In other words, as the medical device 30 is advanced distally, the membrane 20 does not move axially within the vessel 5. The membrane 20 is configured to permit the lumen to radially expand around the medical device 30 as the medical device 30 is advanced through the lumen, as shown in FIGS. 2 and 2B. As the medical device 30 is advanced, the membrane 20 permits the medical device 30 to push the wall radially outward from a central axis of the lumen such that the medical device 30 is permitted to pass through the lumen. In some embodiments, the membrane 20 is configured to permit the lumen to radially expand to a second inner diameter equal to or greater than the maximum outer diameter of the medical device 30. In some embodiments, the membrane 20 is configured to radially expand to a second outer diameter greater than the maximum outer diameter of the medical device 30.

Once the maximum outer diameter of the medical device 30 has moved past a particular axial position along the membrane, the membrane may then constrict inwards toward the central longitudinal axis behind the medical device 30, as shown in FIGS. 2 and 2C. In general, the lumen may maintain a shape or profile that closely conforms to that of the medical device 30 passing therethrough. Accordingly, the membrane 20 may exhibit elasticity in a radial direction with respect to the diameter of the membrane 20 and/or the lumen.

Additionally, while not expressly illustrated, in some embodiments, the membrane 20 may be formed as a mesh, a braid, or a thin-film membrane having a plurality of openings extending laterally or transversely through the wall. The plurality of openings extending through the wall may permit the membrane 20 to contract in the relaxed condition to an even smaller first outer diameter, thereby facilitating use in more tortuous vasculature than a membrane 20 lacking the plurality of openings, while retaining the ability to protect the wall of the vessel 5 from undesirable contact, friction, and shear forces.

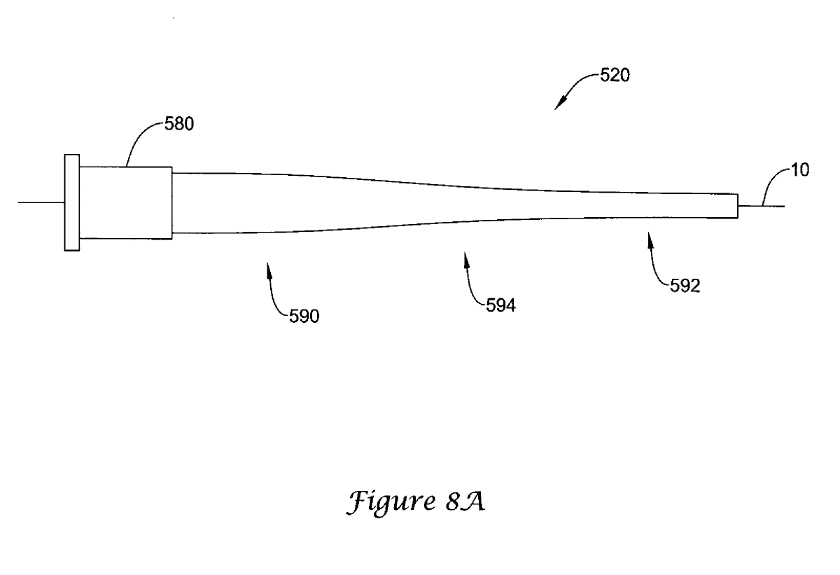

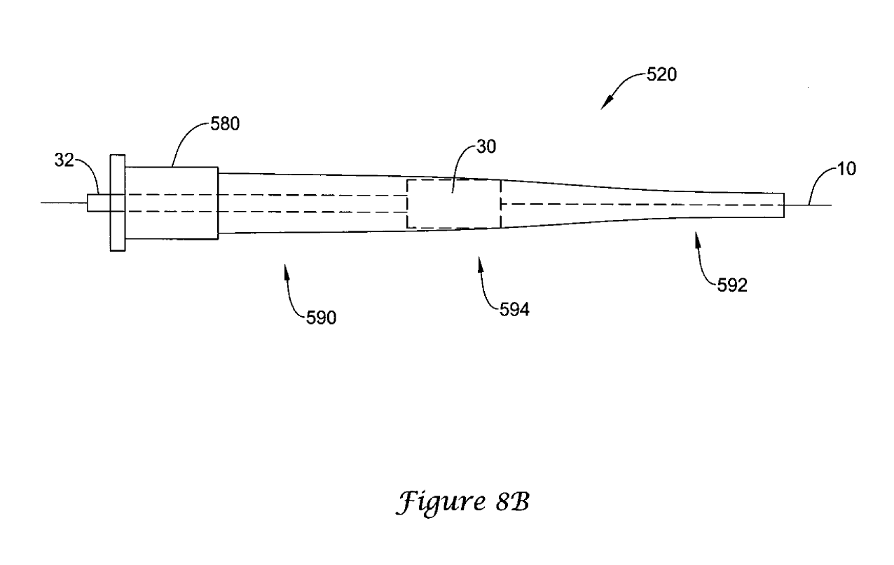

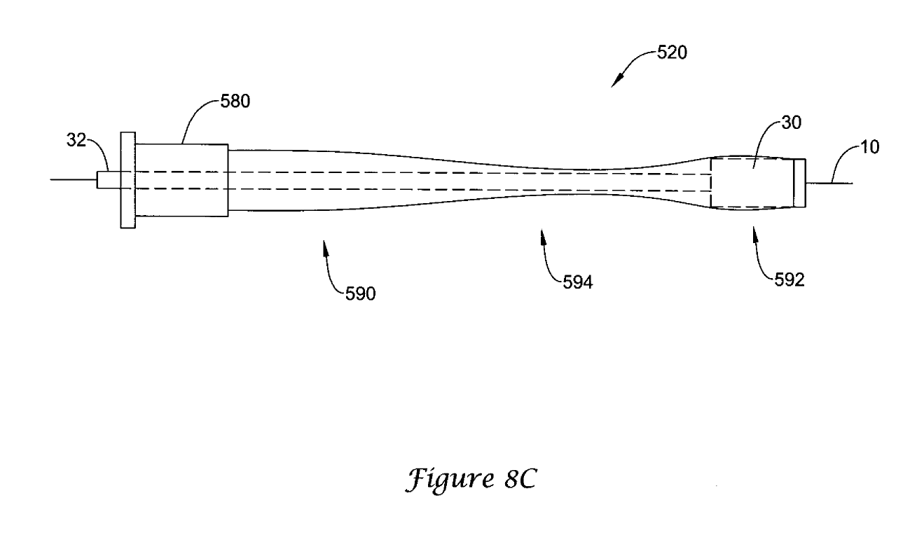

FIGS. 8A-8C schematically illustrate a medical device 30 being advanced through an example membrane 520. In some embodiments, the example membrane 520 may be disposed about or inserted over a guidewire 10, although the guidewire 10 is not required. In some embodiments, the membrane 520 may include a proximal non-expandable section 590 and a distal expandable section 592. In embodiments having a proximal non-expandable section 590, the proximal non-expandable section 590 may have an inner diameter or extent sufficient to accept a medical device 30 passing therethrough, while the distal expandable section 592 may have an inner diameter or radial extent in a relaxed condition that is less than a maximum outer diameter or extent of the medical device 30. The membrane 520 may be formed using any of the techniques or structures discussed herein. Accordingly, the membrane 520 may expand and/or behave similarly to the membrane(s) described above, as similar construction, capabilities, actions, and methods may apply to the membrane 520. As shown, the proximal non-expandable section 590 may taper inward toward the distal expandable section 592 at a central tapered section 594. The central tapered section 594 may provide a transition zone from the proximal non-expandable section 590 to the distal expandable section 592. The central tapered section 594 may be more expandable at a distal end thereof and less expandable at a proximal end thereof with a linear or non-linear transition therebetween. In some embodiments, the membrane 520 may include a proximal manifold or hub 580. FIG. 8B schematically illustrates a medical device 30 (shown in phantom) disposed within the central tapered section 594 of the membrane 520. As the medical device 30 is advanced distally through the proximal non-expandable section 590 and into the central tapered section 594, the medical device 30 may come into contact with the membrane 520, thereby forcing the membrane 520 to expand around the medical device 30 in accordance with the present disclosure. As may be seen from the schematic illustration of FIG. 8C, as the medical device 30 (shown in phantom) advances distally through the central tapered section 594 and the distal expandable section 592, the membrane 520 may contract radially behind the medical device 30.

The guidewire and/or the plurality of fibers may be made from materials such as metals, metal alloys, polymers, ceramics, metal-polymer composites, or other suitable materials, and the like. Some examples of suitable materials may include metallic materials such as stainless steels (e.g. 304 v stainless steel or 316L stainless steel), nickel-titanium alloys (e.g., nitinol, such as super elastic or linear elastic nitinol), nickel-chromium alloys, nickel-chromium-iron alloys, cobalt alloys, nickel, titanium, platinum, or alternatively, a polymeric material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

The membrane and/or the plurality of fibers may be made from materials such as, for example, a polymeric material, a ceramic, a metal, a metal alloy, a metal-polymer composite, or the like. Examples of suitable polymers may include polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials. In some embodiments, a suitable polymeric material may have a yield strain of at least 20%, at least 30%, at least 40%, at least 50%, or more.

Portions of the guidewire, the membrane, and/or the medical device may be made of, may be doped with, may include a layer of, or otherwise may include a radiopaque material. Radiopaque materials are understood to be materials capable of producing a relatively bright image on a fluoroscopy screen or another imaging technique such as X-ray during a medical procedure. This relatively bright image aids the user of device in determining its location. For example, one or more of the elements described above (i.e., the guidewire, the membrane, the medical device, etc.) may include or be formed from a radiopaque material. Suitable materials can include, but are not limited to, bismuth subcarbonate, iodine, gold, platinum, palladium, tantalum, tungsten or tungsten alloy, and the like.

It should be understood that although the above discussion was focused on percutaneous medical procedures within the vasculature of a patient, other embodiments or methods in accordance with the invention can be adapted and configured for use in other parts of the anatomy of a patient. For example, devices and methods in accordance with the invention can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the devices and methods described herein with respect to percutaneous deployment may be used in other types of surgical procedures as appropriate. For example, in some embodiments, the devices may be deployed in a non-percutaneous procedure. Devices and methods in accordance with the invention can also be adapted and configured for other uses within the anatomy.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

1. (canceled)

2. A medical device assembly comprising:
an elongated guidewire;
an elongated tubular membrane disposed about a length of the elongated guidewire and stationary with respect to the elongated guidewire, the elongated tubular membrane having a continuous wall formed from a first material, a proximal end, a distal end, and a plurality of longitudinally-oriented channels recessed along an inner surface of the continuous wall and extending no more than partially through the continuous wall,
wherein the plurality of longitudinally-oriented channels form a wavy pattern or cross-sectional shape,
wherein each channel of the plurality of longitudinally-oriented channels is at least partially filled with a second material different from the elongated tubular membrane to form a surface defining a lumen extending through the elongated tubular membrane from the proximal end to the distal end thereof, the lumen having a first minimum cylindrical inner diameter defined in a relaxed condition by the inner surface of the continuous wall of the elongated tubular membrane and/or the at least partially filled channels of the plurality of longitudinally-oriented channels; and
a percutaneous medical device having a maximum outer diameter greater than the first minimum cylindrical inner diameter of the elongated tubular membrane in the relaxed condition;
wherein the elongated tubular membrane permits the percutaneous medical device to pass over the elongated guidewire and through the lumen of the elongated tubular membrane in a resiliently expanded condition.

3. The medical device assembly of claim 2, wherein the elongated tubular membrane permits the lumen to radially expand to a second inner diameter equal to or greater than the maximum outer diameter of the percutaneous medical device.

4. The medical device assembly of claim 2, wherein the elongated tubular membrane is configured to remain in a substantially stationary position along and about the elongated guidewire as the percutaneous medical device is passed over the elongated guidewire and through the lumen.

5. The medical device assembly of claim 2, wherein the elongated tubular membrane defines an outer diameter that is less than the maximum outer diameter of the percutaneous medical device.

6. The medical device assembly of claim 2, wherein the elongated tubular membrane is configured to substantially prevent axial stretching when the elongated tubular membrane transitions from the relaxed condition to the resiliently expanded condition.

7. The medical device assembly of claim 6, wherein the elongated tubular membrane includes a first plurality of fibers each oriented parallel to an axis of the elongated tubular membrane.

8. The medical device assembly of claim 7, wherein the first plurality of fibers is embedded within the continuous wall of the elongated tubular membrane.

9. The medical device assembly of claim 7, wherein the first plurality of fibers is disposed on a surface of the continuous wall of the elongated tubular membrane.

10. The medical device assembly of claim 2, wherein the plurality of longitudinally-oriented channels permits fluid passage around the percutaneous medical device when the percutaneous medical device is disposed within a portion of the elongated tubular membrane having the plurality of longitudinally-oriented channels.

11. The medical device assembly of claim 2, further including a hemostatic valve disposed within the lumen of the elongated tubular membrane proximal of the distal end.

12. The medical device assembly of claim 11, wherein the plurality of longitudinally-oriented channels extends from the distal end proximally to the hemostatic valve.

13. The medical device assembly of claim 11, wherein the elongated tubular membrane includes one or more apertures disposed through the continuous wall at a location between the distal end and the hemostatic valve.

14. The medical device assembly of claim 2, wherein the continuous wall further includes a lubricious coating disposed on an inner surface thereof.

15. The medical device assembly of claim 2, wherein the percutaneous medical device is selected from the following: an atherectomy device, an angioplasty device, a balloon dilatation catheter, a distal protection device, an embolic filtering device, a valvectomy device, a valvuloplasty device, a stent delivery device, a transaortic valve implantation device, an ablation device, an object retrieval device, a guide catheter or sheath, or a diagnostic catheter.

16. A medical device delivery sheath comprising:
a tubular first layer of polymeric material having a wavy cross-section including a plurality of lobes and a plurality of valleys between adjacent lobes;
wherein the tubular first layer of polymeric material is resiliently expandable in a radial direction from a relaxed configuration to an expanded configuration;
wherein the tubular first layer of polymeric material has a first outer radial extent as measured from a central longitudinal axis in the relaxed configuration, and a second outer radial extent as measured from the central longitudinal axis in the expanded configuration;
wherein the second outer radial extent is greater than the first outer radial extent;
wherein the tubular first layer of polymeric material is substantially non-expandable in an axial direction; and
a tubular second layer of polymeric material disposed on an outside surface of the tubular first layer of polymeric material;
wherein the tubular second layer of polymeric material substantially conforms to the wavy cross-section of the tubular first layer of polymeric material along an inner surface and forms a generally smooth outer surface opposite the inner surface.

17. The medical device delivery sheath of claim 16, wherein the tubular second layer of polymeric material is resiliently expandable in the radial direction from a relaxed configuration to an expanded configuration concurrently with the tubular first layer of polymeric material.

18. The medical device delivery sheath of claim 16, wherein the tubular first layer of polymeric material includes a lower yield strain than the tubular second layer of polymeric material.

19. The medical device delivery sheath of claim 16, further including a tubular third layer of polymeric material disposed on an inside surface of the tubular first layer of polymeric material;
wherein the tubular third layer of polymeric material substantially conforms to the wavy cross-section of the tubular first layer of polymeric material along an outer surface and forms a generally smooth inner surface opposite the outer surface.

20. A medical device assembly comprising:
an elongated guidewire;
an elongated tubular membrane initially disposed about a length of the elongated guidewire, the elongated tubular membrane having a continuous wall defining a lumen extending through the elongated tubular membrane from a proximal end to a distal end, the lumen having a first inner diameter defined in a relaxed condition by the inner surface of the continuous wall; and
a percutaneous medical device having a maximum outer diameter greater than the first inner diameter of the elongated tubular membrane in the relaxed condition;
wherein the elongated tubular membrane permits the percutaneous medical device to be advanced at the distal end of an elongate shaft along and about the elongated guidewire and through the lumen in a resiliently expanded condition;
wherein the elongated tubular membrane includes a plurality of longitudinally-oriented channels recessed along an inner surface of the continuous wall, the plurality of longitudinally-oriented channels extending no more than partially through the continuous wall and forming a wavy pattern or cross-sectional shape,
wherein the plurality of longitudinally-oriented channels are filled with a material different from the elongated tubular membrane to form with the elongated tubular membrane a smooth surface of the first inner diameter in a relaxed condition.
